(19) United States
(12) Patent Application Publication
(10) Pub. No.: US 2025/0045854 A1
(43) Pub. Date: Feb. 6, 2025

(54) Anonemis Research Software Hard Drive Protocol Policy Processes 2305

(71) Applicant: Renee Simenona Martinez, Beverly Hills, CA (US)

(72) Inventor: Renee Simenona Martinez, Beverly Hills, CA (US)

(73) Assignee: Renee Simenona Martinez, Beverly Hills, CA (US)

(21) Appl. No.: 18/230,148

(22) Filed: Aug. 3, 2023

(51) Int. Cl. G06Q 50/26; G01N 33/50; G06T 7/00

(52) U.S. Cl. CPC G06Q 50/26; G01N 33/50; G06T 7/0014

(57) ABSTRACT

In this embodiment, an algorithmic software process and isolated observation pattern recognition system are used to monitor micropatterns of the subject or content in order to identify and correct miscalculated media content, processes, sequences, operations, or actions. In order to reconstruct the corrected pattern, the Quantum Parallel Data Algorithm Structure is used. The infrastructure is designed to operate as an encryption system that runs simultaneously but perpendicularly as a single method for every transaction in conjunction with AR Quantum Sequence Triplication with Quantum Operation Supremacy for final accuracy testing.

### FIELD

The present application relates to the embodiment compromising of a software application process in quantum science regarding specialized algorithmic methodology procedure with critical outcome processes, as it relates to a specialized encrypted algorithmic quantum process procedure. The stated relates to a software application and isolated methodology procedures, as it relates to correction and various tactical areas. This software application includes a user-interface with a customized operating system.

This software application encompasses a customized operating system which yields to areas of telecommunications, automotives, transliteration development, chip technology, communications, data analysis, data algorithm structure, mechanical engineering, behavioral science, medicine, behavioral science, nuclear science, space technology, defense, quantum approximation and any software devices offering access to information.

### BACKGROUND

AR Quantum Parallel Data Algorithm Structure is an embodiment comprising of a specialized algorithmic software process. The stated software process is a multioperation system which runs on a quantum software process and methodology which data source references are critically verified information by Quantum Sequence Triplication.

Implementing Quantum Parallel Data Algorithm Structure operates by shuffling at a rapid pace on the server system to complete each verified transaction to ensure accuracy during the process. This infrastructure operates as an encryption which runs simultaneously but perpendicularly as a single method for every transaction in connection with AR Quantum Sequence Triplication with Quantum Operation Supremacy for final accuracy testing.

In addition to utilizing auto-populated encryption infrastructure to ensure security, accuracy and assuring all configurations are current and up to date within the standard and practices. Implementing randomized auto-penetration testing throughout the workday to ensure the responsiveness and effectiveness of the security infrastructure in place within the department.

### SUMMARY

This embodiment of the compromising software process regarding subject or contents to ensure that miscalculations in media content, processes, sequences, operations, or actions are corrected with a critical interface includes an encrypted algorithm which records micropatterns of isolated content, and essential elements of tactical process procedures within various sectors stated within present application, which requires Quantum Parallel Data Algorithm Structure is to achieve such computation.

The stated Algorithm Structure operates by shuffling at a rapid pace on the server system to complete each verified transaction to ensure accuracy during the process. This infrastructure operates as an encryption which runs simultaneously but perpendicularly as a single method for every transaction in connection with AR Quantum Sequence Triplication with Quantum Operation Supremacy for final accuracy testing.

The use 3 qubit bits to achieve quantum supremacy without grey area bias. This feature is essential for monitoring intrusions or malfunctions in a multitude of systems in various sectors. The rapid shuffling process is a specialized technique which allows swift detection in any mechanism, process procedure, or operation.

### BRIEF DESCRIPTION OF THE DRAWINGS

In the following detailed portion of the present description, the teachings of the present application will be explained in more detail with reference to the example embodiments shown in the drawings, in which:

FIG. 1. Quantum Parallel Data Algorithm Structure

FIG. 2. Blu Vision Lense

FIG. 3. AR Solar Development

FIG. 4. AR Solar Harvest Energy

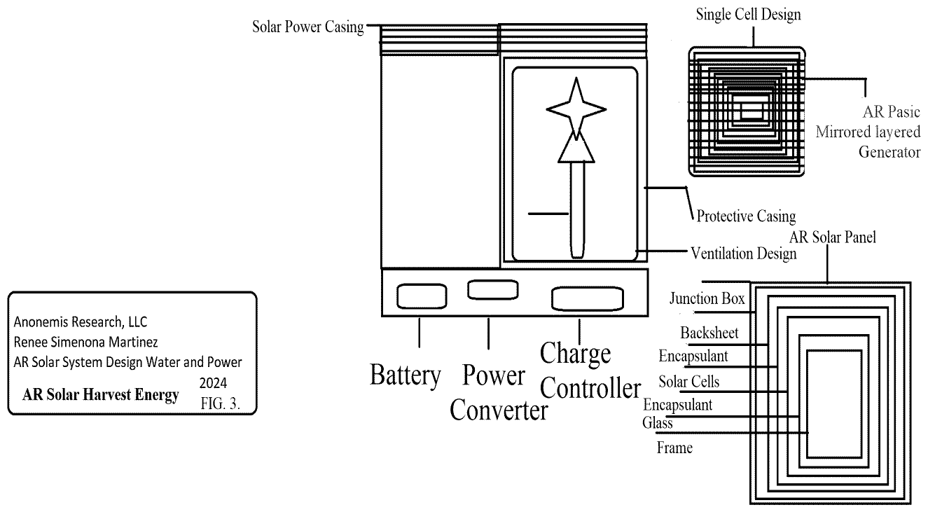

FIG. 5. Chip-Ray Reflector Reproduction Simulation Trapping Ions

### DETAILED DESCRIPTION

In the following detailed description, the software process according to the teachings for this application in the form of technical science will be described by the embodiments. It should be noted that the teachings of this application can also be used in any other sectors such as: telecommunications, automotives, transliteration development, solar, chip technology, communications, data analysis, behavioral science, transportation, medicine, nuclear science, space technology; defense, quantum approximation, data algorithm structure, mechanical engineering and any software devices offering access to information.

The first embodiment comprising of stated system that works by implementing a specialized rapid algorithm process of monitoring micro-patterns of a subject or contents to ensure that miscalculations in content, processes, sequences, operations, or actions are corrected by Quantum Sequence Triplication. The stated software process operates on a single quantum system, therefore allowing the optimal sequencing and tactical outcome.

Quantum Parallel Data Algorithm Structure operates by shuffling at a rapid pace on the server system to complete each verified transaction to ensure accuracy during the process. This infrastructure operates as an encryption which runs simultaneously but perpendicularly as a single method for every transaction in connection with AR Quantum Sequence Triplication with Quantum Operation Supremacy for final accuracy testing.

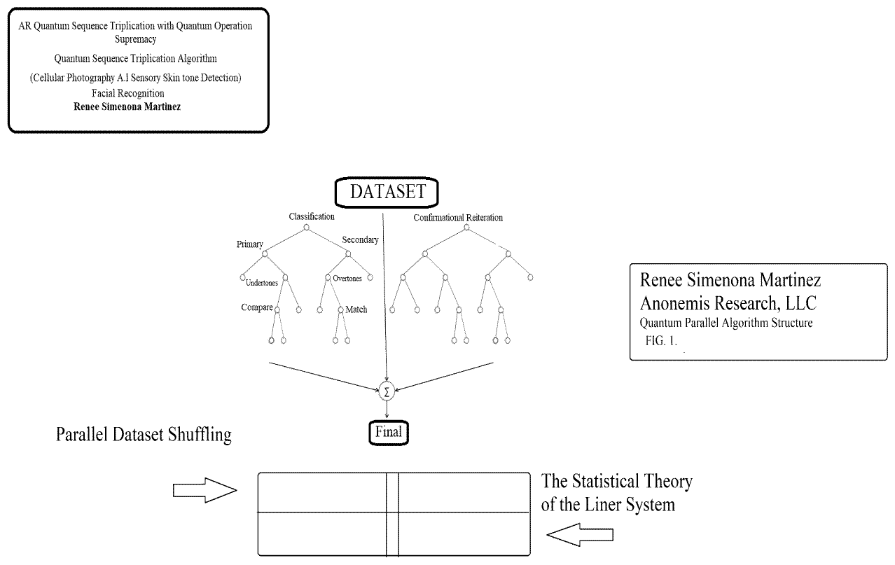

This embodiment comprises of the use behavioral isolation observation and specialized algorithm to determine computation results. As illustrated in FIG. 1., AR Deep Imaging Perception Camera Imaging. This is a series of advancements in camera lens technology which can be utilized in Digital SLR's as well as cell phone cameras. The purpose of this Imaging System is to give the users enhanced experience providing for increased detail and depth as well as 3D photos, and 360-degree object views. This is the re-invention of photography as we know it. The enhanced imagery will be translated into printable photos as well: think 3D polaroids.

Expanded Deep Imaging. Utilizing highly specialized light bending mirrors within the lenses, the technology successfully bends light around the object using this series of strategically placed mirrors. This allows the light to dance around the object to increase the detail and depth, providing a 3D image that will pop off the viewfinder as well as the screen. This of course works in tandem with 3D software built into phones and computers. This process is completed by behavioral environment isolation monitoring.

AR 360 Deep Imaging. Utilizing the panoramic, continuous capture already available in many cameras and phones, the user can circumvent the object to create an image that will allow viewers to rotate the object and view it from all angles in editing. A common use of this feature would be in portrait photography.

For example: a photographer could take a 360 view of the model and rotate the image to produce the best possible photo, choosing to feature either the right side or the left side of the model's face. This feature can also be used in selfie mode.

Another feature will include dual extendable mirrored lenses which serve to enhance the depth and detail of images by providing increased angles and lighting while the object is stationary.

This also enhances the 360 Deep Imaging when the user is unable to use the circumvent panorama feature. Contrast Depth Reimaging. Fascinated how deep fakes provide depth to film. Imagine this image smoother and with contrasting surroundings to give it that extra sense of realism. It's about adding textures to pivotal objects that help merge the viewer into the experience as one. It's different than virtual reality because it requires no additional accessories to capture. It changes the movie completely, the entire experience.

The use of deep fakes superimposed faces onto people, we can use this method to create more immersive viewing. Imagine action films, using the same technology but on animals or explosions. This innovation allows the replacement of previous actors in films with new ones. This allows users to exchange actors within any film and television for a customized viewing experience.

This technology is essential for AR Hyperrealism with skin 360 criminal identification illustration to better help identify criminals and missing persons.

This embodiment comprises of, compromising of AR Blu Vision Lense which contains a stream of Social with a visual mouse control by biometric detection. Eye protection support for screen and spatial detection and orientation to prevent obstructed vision. Email scanner visual mouse control and audio read and filtered. The options to find other players and different platforms. Immersive World Generator Specs: Speaker Glass Frame Temples, Visual Mouse Control, TV and Projection, Transition Lens Engagement, Bridge expansion on top bar and peripheral vision engagement on end piece of glasses. Lastly, 360 View, Sleek Minimized and non-detractive interface.

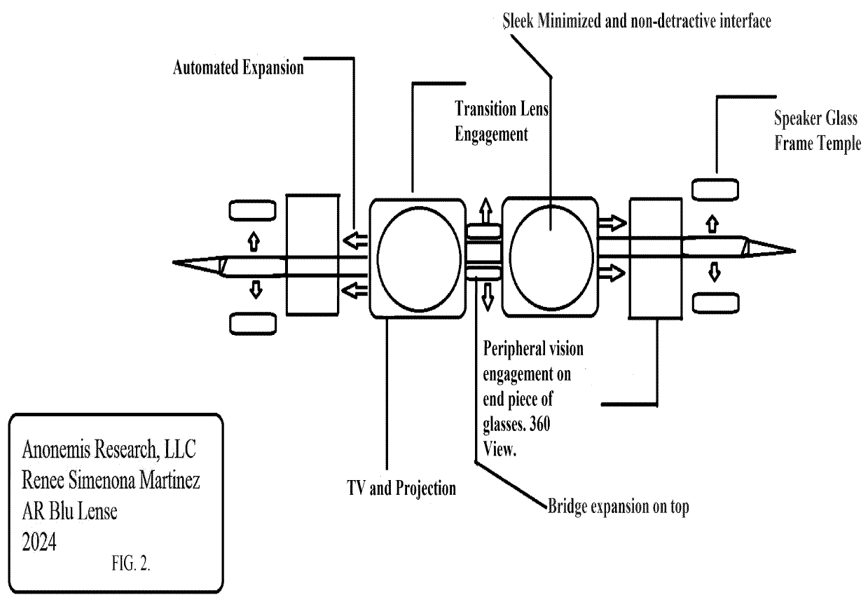

The embodiment comprises a specialized algorithm to determine computation results in AR Solar Development, as shown in FIG. 2. AR Solar Water-Cooling Vapor: The solar water-cooling process inherently produces condensate water. As warm air traverses the evaporator coils, it undergoes cooling, causing moisture within the air to condense.

This condensed water gathers in a drip pan and is typically channeled away. On average, a unit yields approximately 3 to 4 gallons of water per hour, with the exact amount contingent upon the size and efficacy of the AC unit. Larger or more efficient units may generate up to 10 gallons per hour. Solar Water-cooling Vapor incorporates all 4 systems to ensure the best outcome.

### Side Stream Filtration for Cooling Towers

Side stream filtration systems continually filter a portion of the cooling water to eliminate suspended solids, organics, and silt particles. This process mitigates fouling, biological growth, scaling, and corrosion within cooling towers.

Centrifugal Separators: Devices that utilize centrifugal force to separate solids from water.

Automatic Screen Filters: Implement screens to capture particles.

Plastic Disc Filters: Utilize plastic discs with fine openings to ensnare impurities.

Sand Filters: Pressure Sand Filters: Employ sand beds to filter water.

High Efficiency Sand Filters: Enhance filtration efficiency.

### Chiller Water Filtration

Chiller systems encompass a condenser loop comprising an outdoor cooling tower, pump, condenser coil (inside the chiller), and related piping.

### Cooling Tower Filtration

Cooling towers serve dual functions:

Temperature Reduction: They draw outside air and water into the tower, where some water evaporates, thereby cooling the remaining recirculated water within the system.

AR Dual Split Filtration: This system is a dual filtration system which classifies two types of stored water: Drinking and Outside use.

These filtration systems significantly contribute to operational efficiency, lifespan, and overall performance across various industrial and environmental settings. This software process and device is operated by stated algorithm,

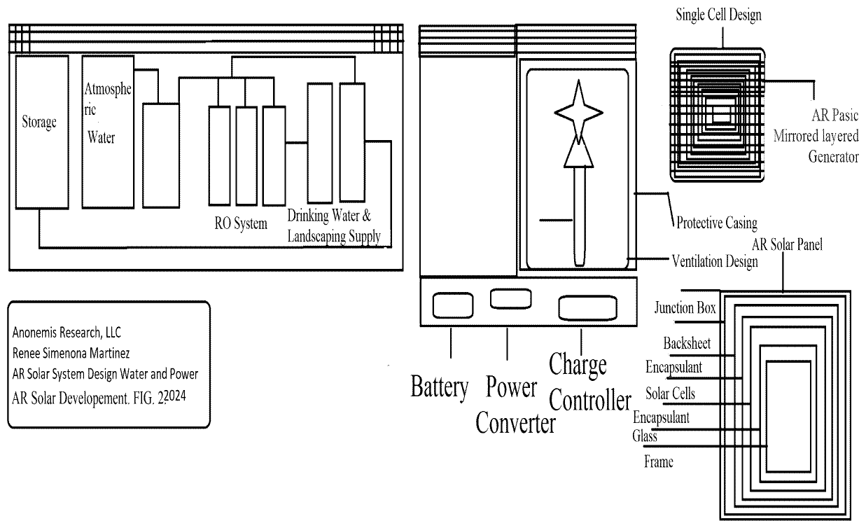

The embodiment comprising of AR Solar Harvest Energy: As illustrated in FIG. 3., A wind-solar hybrid system represents a notable alternative power generation configuration amalgamating the strengths of wind and solar energy sources. Let's explore the specifics:

### Advantages of Hybrid Systems

Enhanced Energy Production: By harnessing both wind turbines and photovoltaic (solar) panels, these hybrid systems optimize energy generation.

Complementary Power: In regions characterized by fluctuating weather patterns, the amalgamation of wind and solar power ensures a more consistent electricity supply.

Operational Flexibility: Hybrid systems can function off-grid, disconnected from the conventional electricity distribution network. During periods of inadequate wind or solar power, most hybrid setups rely on batteries and/or engine generators powered by fuels such as diesel. These backup systems activate to furnish electricity and recharge batteries as necessary. Storage Capacity: Battery banks are typically dimensioned to supply electric loads for one to three days, guaranteeing uninterrupted power during non-charging intervals.

How It Works: Solar System: Solar panels capture sunlight energy during the day, particularly in summer.

Wind Turbines: These turbines generate power from the air, predominantly during nighttime and winter months.

Quantum Parallel Data Algorithm Structure allows detection and automatic correction on remote software.

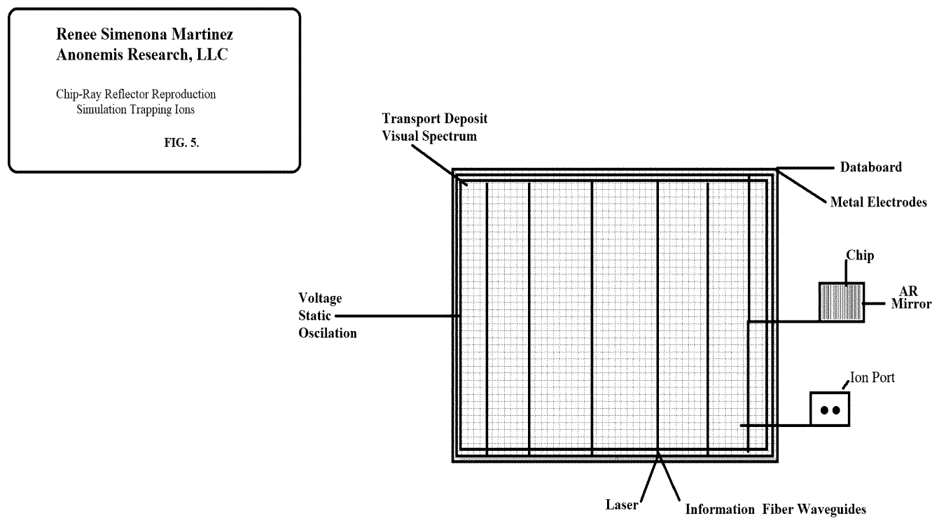

The embodiment comprising of Chip-Ray Reflector Reproduction Simulation Trapping Ions (C.R.R.R.S.T) is an integration technology developed to address a large scale of ion qubits. C.R.R.R.S.T traps the ion and holds them in a fixed position by a combination of voltages, both static and oscillating, that are applied to metal electrodes placed around the positions we'd like the ions to be trapped in on the squared chip-ray reflector, as shown in FIG. 5.

The ions are trapped above the surface of the plane in the C.R.R.R.S.T board, with the surface to ion distance scale being set by the size and spacing of the electrodes. Planar electrodes can be fabricated using microchip and mirror projecting techniques, thus, producing large numbers of zones arrayed in two dimensions for large numbers of ions to be trapped in, as well as to reduce the size of the electrodes to the micrometer scale.

The optics on a chip, tiny fiber waveguides are made by depositing the right materials, which of course have to be transparent over the visible spectrum, onto the trap chips. These materials are then patterned on millions of square units which are both physically and artificially integrated by the use of reflective ray reproduction, using the same techniques used to pattern the ion trap electrodes.

These waveguide patterns define the paths that the laser light travels along, and we can design them to split the light from one path into many branches. The waveguides for the laser are a vertical layout for each board in the tower, designed to trap one ion per laser. The boards are double faced allowing enough for trap four ions per ion-chip-ports by pattern gaps in the waveguide material that act like a diffractive grating.

AR Quantum Parallel Data Algorithm Structure run in conjunction with this specialized software process.

The invention claimed is:

1. An algorithmic software and isolated observation pattern recognition system is used in this embodiment to monitor the micropatterns of the subject or content to identify and correct media content, process, sequence, operations, or actions that have been miscalculated. The corrected pattern is then reconstructed using the Quantum Parallel Data Algorithm Structure. This infrastructure operates as an encryption which runs simultaneously but perpendicularly as a single method for every transaction in connection with AR Quantum Sequence Triplication with Quantum Operation Supremacy for final accuracy testing.

1. A software process and methodology according to claim 1, wherein compromising an algorithmic software and isolated observation pattern recognition system used in this embodiment to monitor the micropatterns of the subject or content to identify and correct media content, process resulting in Solar Satellite Telecommunications Network and the Solar Transportation Network with the incorporation of live digital mapping system displaying electrical, satellite, and solar power sources to established, activity, precise location in the event of an outage and potentially where a redirecting of power sourcing can be used. It is essential for cities to have a redirecting resource in place regarding its transportation, power and telecommunications networks. It is critical to have a multi-system plan in motion.
